(19) United States
(12) Patent Application Publication
(10) Pub. No.: US 2008/0078406 A1
(43) Pub. Date: Apr. 3, 2008

(54) Endotracheal tube and technique for using the same

(76) Inventors: Jessica Clayton, Campbell, CA (US); Paul W. Martens, Pleasanton, CA (US); Michael P. O'Neil, Pleasanton, CA (US)

Correspondence Address: Covidien;IP Counsel - Respiratory & Monitoring Solutions, 60 Middletown Avenue, North Haven, CT, 06473, US

(21) Appl. No.: 11/541,491

(22) Filed: Sep. 29, 2006

(51) Int. Cl. A61M 16/00

(52) U.S. Cl. 128/207.15

(57) ABSTRACT

There is disclosed an endotracheal tube to which an adhesion-resistant material, adhesion-resistant material is applied. Several techniques are disclosed for applying the adhesion-resistant material, adhesion-resistant material, including surface treatments, co-extrusion, and compounding. The adhesion-resistant material, adhesion-resistant material helps prevent the adhesion of microbes to the surface of the endotracheal tube. In this manner the cross-sectional area through which the patient may breathe is increased, effectively decreasing the work of breathing for the patient.

### BACKGROUND OF THE INVENTION

1. Field of the Invention

The present invention relates to medical devices, and more particularly, to airway devices, such as tracheal tubes.

2. Description of the Related Art

This section is intended to introduce the reader to various aspects of art that may be related to the present invention which is described and/or claimed below. This discussion is believed to be helpful in providing the reader with background information to facilitate a better understanding of the various aspects of the present invention. Accordingly, it should be understood that these statements are to be read in this light, and not as admissions of prior art.

In the course of treating a patient, a tube or other medical device may be used to control the flow of air, food, fluids, or other substances into and/or out of the patient. For example, medical devices, such as tracheal tubes, may be used to control the flow of one or more substances into or out of a patient. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

Tracheal rubes may be used to control the flow of air or other gases through a patient's trachea. Such tracheal tubes may include endotracheal tubes, and tracheostomy tubes. To seal these types of tracheal tubes, inflatable cuffs are sometimes associated with these tubes. When inflated, these cuffs generally expand into the surrounding trachea to seal the tracheal passage around the circumference of the tube. A high-quality seal against the tracheal passageway allows a ventilator to perform efficiently.

Generally, patients are ventilated under positive pressure conditions. This generally means that the inspired air is pushed into the lungs by the ventilation device, which is a passive process for the patient, while the expired air is pushed out by the patient's lungs. In this context, the airway resistance is the amount of effort required by the patient to exhale through an endotracheal tube. Thus, any increase in the work of breathing can negatively impact the comfort of the patient. Keeping a patient's work of breathing to a minimum also facilitates a patient being weaned off ventilation and effectively decreases the time that patient may be intubated.

A buildup of material on the inside of the tube may increase the resistance to the flow of air through the tube, thereby increasing the work of breathing for the patient. One contributor to such an increase may be the build-up of mucus on the inside of the endotracheal tube. For example, a patient's coughing may dislodge mucus in the lungs and cause it to back up into the tube. Further, the build-up of mucus may facilitate the formation of biofilms on the inside surface of the tube. Mucus may not only contain the microbes that may form biofilms, but may also present a surface on the inside of the tube that may encourage the formation of biofilms. Also, the adhesion of biofilms to the may in turn encourage mucus to build up inside the tube. Additionally, biofilm formation is generally not desirable, as biofilms may be related to certain clinical complications.

Various techniques have been employed to prevent the accumulation of mucus or other buildup on the inside wall of an endotracheal tube. These include: introducing humidified air into the tube to thin the mucus and reduce the build-up, utilizing a device to scrape clean the inside of the tube, and inserting a suction catheter to vacuum the mucus from the walls. However, despite the now-common utilization of humidified air, the problem of mucus on the tube walls has not been eliminated. Further, the use of the scraping device and suction catheter involves introducing an apparatus within the tube and operating the device in vivo. This is time-consuming for the technician operating these devices. Additionally, these devices lack any feedback to ensure the tube has been sufficiently cleaned. In addition, these various techniques, alone and in combination, do not provide a preventive solution for the buildup of microbes that does not involve regular maintenance. A device for minimizing the build-up of microbes in endotracheal tubes that is non-invasive and preventative in nature is therefore desirable.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

There is provided an endotracheal tube that includes: a conduit that includes an inner surface defining a fluid passageway; an adhesion-resistant material that is resistant to microbial adhesion disposed on at least a portion of the inner surface, wherein the adhesion-resistant material has a water contact angle of less than 40 degrees; and an inflatable cuff disposed on the conduit.

There is also provided a method of manufacturing an endotracheal tube that includes: providing a conduit having an inner surface defining a fluid passageway; providing an adhesion-resistant material that is resistant to microbial adhesion disposed on at least a portion of the inner surface, wherein the adhesion-resistant material has a water contact angle of less than 40 degrees; and providing an inflatable cuff disposed on the conduit.

There is also provided a method of decreasing microbe adhesion to an endotracheal tube that includes: inserting an endotracheal tube into a patient, wherein the endotracheal tube includes a conduit including an inner surface defining a fluid passageway; and applying an adhesion-resistant material on at least a portion of the inner surface, wherein the adhesion-resistant material is adapted to prevent microbe adhesion and wherein the adhesion-resistant material has a water contact angle of less than 40 degrees.

There is also provided an endotracheal tube kit that includes an endotracheal tube that includes: a conduit including an inner surface defining a fluid passageway; an inflatable cuff disposed on the conduit; and a lumen operatively connected to the inner surface of the conduit, wherein the lumen is adapted to dispose an adhesion-resistant material that is resistant to microbial adhesion on the inner surface of the conduit, wherein the adhesion-resistant material has a water contact angle of less than 40 degrees; and a syringe including the adhesion-resistant material, wherein the syringe is adapted to be operatively connected to the lumen.

### BRIEF DESCRIPTION OF THE DRAWINGS

Advantages of the invention may become apparent upon reading the following detailed description and upon reference to the drawings in which:

FIG. 1 illustrates an exemplary endotracheal tube including an adhesion-resistant material on an inner surface;

FIG. 2 illustrates a view of an exemplary endotracheal tube inserted into a patient's trachea;

FIG. 3 illustrates a cross-sectional view of the tube core material with an interior adhesion-resistant material layer applied;

FIG. 4 illustrates a cross-sectional view of the tube core material with an interior and exterior adhesion-resistant material layer applied;

FIG. 5 illustrates a cross-sectional view of the tube core material compounded with the adhesion-resistant material to form a monolayer; and

FIG. 6 illustrates an exemplary endotracheal tube including a lumen adapted to deliver the adhesion-resistant material to an inner surface.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

It is desirable to provide a medical device that is resistant to the buildup of mucus or other materials that offers a non-invasive buildup resistance mechanism that does not involve regular maintenance. In accordance with some aspects of the present invention, an endotracheal tube is provided that includes an adhesion-resistant material adapted to reduce mucus and/or microbe adhesion to the surfaces of the tube. This adhesion-resistant material may function to prevent the accumulation of mucus on the interior surface of the endotracheal tube, thereby maximizing the cross-sectional area within the tube through which the patient may breathe. Additionally, in certain embodiments, the lubricious qualities of the adhesion-resistant material may also decrease the coefficient of friction of fluids, such as gas, being transferred in the tube. This, in turn, may function to minimize the patient's work of breathing, positively impacting the comfort of the patient. Further, such endotracheal tubes may also provide the advantage of reduced microbe adhesion and/or buildup on the surfaces of the tube, which may reduce related clinical complications. For example, many types of bacteria adhere more readily to hydrophobic surfaces. Thus, reducing the hydrophobicity of the endotracheal tube's fluid passageway may make the tube more adhesion-resistant to microbes. Preventing the initial adhesion of microbes to the inside surface of the tube may in turn prevent or reduce biofilm formation on the inside surface of the tube.

The adhesion-resistant materials may be used in conjunction with any suitable medical device. In certain embodiments, the adhesion-resistant materials as provided herein may be used in conjunction with a catheter, a stent, a feeding tube, an intravenous tube, an endotracheal tube, a tracheostomy tube, a circuit, an airway accessory, a connector, an adapter, a filter, a humidifier, a nebulizer, or a prosthetic.

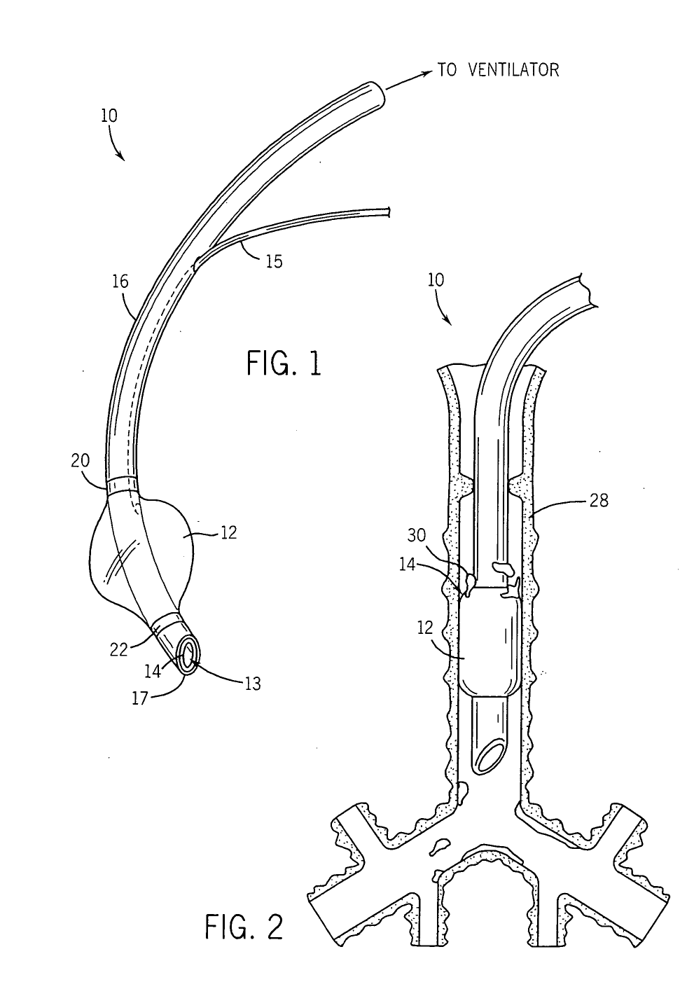

An example of a medical device including an adhesion-resistant material is an endotracheal tube 10, as depicted in FIG. 1. The endotracheal tube 10 includes an inflatable cuff 12 and a adhesion-resistant layer 14 disposed on the fluid passageway 13 defined by a conduit 16. The conduit 16 is suitably sized and shaped to be inserted into a patient to allow the passage of air through the conduit 16. Typically, the inflatable cuff 12 is disposed, adhesively or otherwise, towards the distal end 17 of the conduit 16. The cuff 12 may be inflated and deflated via a lumen 15 in communication with the cuff 12, typically through a hole or a notch in the conduit 16. The cuff 12 has a proximal opening 20 and a distal opening 22 formed in the cuff walls to accommodate the conduit16.

The adhesion-resistant layer 14 may be disposed on all or a portion of the inner surface of the conduit 16. For example, in certain embodiments, it may be advantageous dispose the adhesion-resistant layer 14 towards the distal end 17 of the conduit 16, as mucus being coughed into the conduit 16 from the lungs may first enter the distal end 17. Generally, the adhesion-resistant layer 14 may be sufficiently thick to cover an inner surface of the conduit 16 while not being so thick as to significantly impact the flow of fluid through the fluid passageway 13. In certain embodiments, the adhesion-resistant layer 14 may be less than 1 mm thick. For example, an extruded layer may be 0.0001 inches in thickness. It may also be advantageous to employ a nonswellable adhesion-resistant layer 14 in order to minimize the effect of the adhesion-resistant layer 14 on the inner diameter reduction of the fluid passageway 13. However, a swellable adhesion-resistant layer 14 may be used in which any swelling is limited or constrained, or in embodiments in which the layer 14 is sufficiently thin that swelling will have a negligible effect on the inner diameter of the conduit 16.

In one embodiment, the adhesion-resistant layer 14 may be characterized by its degree of hydrophilicity. A hydrophilic adhesion-resistant layer 14 may be advantageous, as many types of bacteria adhere more readily to hydrophobic surfaces. One such measure of hydrophilicity is a contact angle measurement, done by, for example, the sessile drop method. On hydrophilic surfaces, a water droplet will spread out over a larger area than on a hydrophobic surface. The contact angle is the angle at which a liquid/vapor interface meets the solid surface. The shape of the droplet may be determined by the Young-Laplace equation. On many hydrophilic surfaces, water droplets will exhibit contact angles of 0° to 40°. For example, certain hydrogels may be so hydrophilic that water disappears on their surfaces. Such materials may be considered to have a water contact angle of zero. On hydrophobic surfaces, which are resistant to water, one observes a large contact angle (70° to 90°). Thus, the adhesion-resistant layer 14 may have a water contact angle of less than 40° or between 10° to 30°. It should be understood that a generally hydrophilic material, such as a polyethylene glycol, may also include hydrophobic elements, such as a hydrophobic backbone. Further, in certain embodiments the hydrophilic surface may have surface chemistries which provide surface energies not favorable for deposition (for example surface treatments to covalently bind hydrophilic compounds, including ammonia, oxygen, proteins or polysaccharides to the surface), and also a physical steric hindrance effect, where polymer/oligomer chains make it difficult for microbial adhesion to occur. Thus, highly branched hydrophilic materials, such as polyethylene glycols, may be advantageous for use as adhesion-resistant materials.

In certain embodiments, the adhesion-resistant layer 14 may be characterized by its coefficient of friction against the flow of gas through the conduit 16. Generally, the adhesion-resistant layer 14 may exhibit decreased friction and resistance to gas flow as compared to the relatively hydrophobic material of the conduit 16. Airway resistance is the opposition to gas flow caused by the forces of friction. Resistance to flow in the airways depends on whether the flow is laminar or turbulent, the dimensions of the airway, and the viscosity of the gas. For laminar flow, resistance is quite low. That is, a relatively small driving pressure is needed to produce a certain flow rate.

In other embodiments, the adhesion-resistant layer 14 may be characterized by the adhesion-resistant material from which it is formed. For example, the adhesion-resistant layer 14 may include polyethylene glycols (e.g. BASF Pluronics F-127), polyethylene oxides, polyvinyl alcohols (e.g. Supersorb ionic vinyls), polyalkylene glycols, alkoxy polyalkylene glycols, polysaccharides, polyvinylpyrrolidones, polyacrylic acids, polyacrylamides, polymaleic anhydrides, or copolymers thereof and mixtures thereof. In embodiments in which the material used to form the adhesion-resistant layer is insufficiently hydrophilic, a plasma treatment may be employed to alter the surface chemistry of the adhesion-resistant layer so that its water contact angle is less than 30°.

The cuff 12 may be formed from materials having suitable mechanical properties (such as puncture resistance, pin hole resistance, tensile strength), chemical properties (such as forming a suitable bond to the tube 16), and biocompatibility. In one embodiment, the walls of the inflatable cuff 12 are made of polyurethane having suitable mechanical and chemical properties. An example of a suitable polyurethane is Dow Pellethane® 2363-80A. In another embodiment, the walls of the inflatable cuff 12 are made of a suitable polyvinyl chloride (PVC). Suitable materials may also include polyethylene teraphthalate (PETP), low-density polyethylene (LDPE), polypropylene, silicone, neoprene, or polyisoprene. Typically, endotracheal cuffs are inflated within a patient's trachea such that the intra cuff pressure is approximately 20-30 cm H₂O. Endotracheal cuffs utilizing inflation pressures significantly greater than 25 cm H₂O, such as 100 cm H₂O, may be referred to as high-pressure cuffs, while cuffs that are designed to be inflated at pressures less than 25 cm H₂O may be considered low-pressure cuffs.

FIG. 2 shows the exemplary endotracheal tube 10 that has been inserted into a patient's trachea. The cuff 12 is inflated to form a seal against the tracheal walls 28. In addition to isolating the passageway 13, the cuff 12 may prevent secretions 30 or other detritus from passing through the trachea downward into the lungs. However, mucus buildup in the bronchus or lungs may be dislodged and propelled upward into the distal end of the conduit 16. The adhesion-resistant layer 14 may prevent the mucus from adhering to the fluid passageway 13 of the conduit 16, allowing the fluid, such as a respiratory gas mixture, to be transferred without substantial change in the conduit inner diameter.

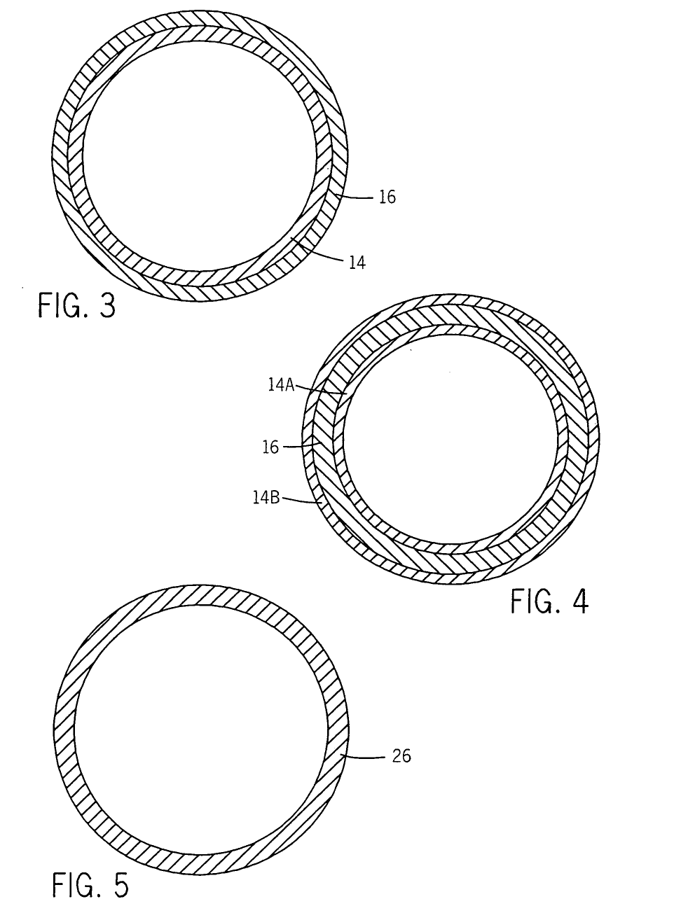

The adhesion-resistant layer 14 may be manufactured and applied to the cuff 12 by any suitable technique. For example, the adhesion-resistant layer 14 may be co-extruded with the conduit 16. FIG. 3 depicts a representative cross-section of any portion of the conduit 16 and an exemplary co-extruded adhesion-resistant layer 14 resulting from this process. The co-extruder may include two concentric extrusion dies, which may by fed polymer pellets having particular characteristics to form the conduit 16 and the adhesion-resistant layer 14. The extruder may melt the polymers to feed the molten polymer through the dies to form a double-layered tube shape. In one embodiment, a programmable parasin may be used to specify the thickness of the adhesion-resistant layer 14.

In other embodiments, the adhesion-resistant layer 14 may be applied to the conduit by radio frequency-oxygen (RF—O₂) glow discharge or plasma treatments. Such techniques may be particular useful in embodiments in which the adhesion-resistant layer 14 is substantially thinner than the conduit 16. For example, an adhesion-resistant layer 14 may be only a few microns in thickness when applied by a radio frequency-oxygen (RF—O₂ glow discharge technique) or any other appropriate plasma or chemical vapor deposition surface treatment. In certain embodiments, the adhesion-resistant layer may also include an adhesion layer or a tie layer. As shown in FIG. 4, an adhesion-resistant layer 14a and 14b may also be applied to both the interior and exterior surfaces of the conduit 16. The adhesion-resistant layers 14a and 14b may also be an extruded or co-extruded layer. In other embodiments, the interior and exterior adhesion-resistant layers 24 may be formed by dipping the conduit 16 in an adhesion-resistant material.

In other embodiments, as shown in FIG. 5, an adhesion-resistant material may be compounded into the core material of the conduit 16, resulting in a single adhesion-resistant material monolayer 26. This embodiment may result from mixing or compounding the adhesion-resistant material with the conduit polymer, and then carrying out any of the extrusion processes discussed previously for forming the endotracheal tube 10.

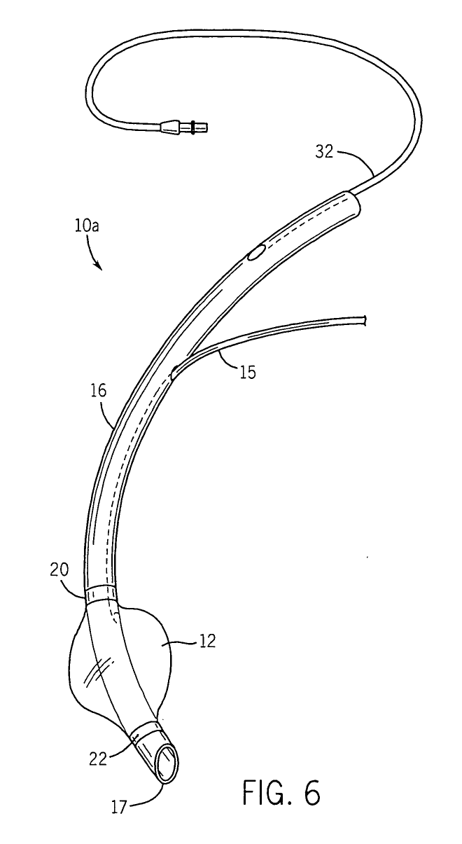

FIG. 6 illustrates an alternative endotracheal tube 10a that includes a lumen 32 suitably sized and shaped to deliver an adhesion-resistant material to the interior surface of a conduit 16. For example, the adhesion-resistant material may be carboxycellulose. In a specific embodiment, the lumen 32 may be adapted to initially deliver an adhesion-resistant material to the passageway 13 of the conduit 16, and it may also subsequently deliver additional amounts of the adhesion-resistant material as needed. For example, as part of routine endotracheal tube 10 maintenance, an adhesion-resistant material may be reapplied to the fluid passageway 13. In certain embodiments (not shown), an endotracheal intubation kit may include a cuffed endotracheal tube 10a and a prefilled syringe (not shown) including an appropriate adhesion-resistant material.

The endotracheal tube 10 of the present invention may be incorporated into systems that facilitate positive pressure ventilation of a patient, such as a ventilator. These systems may include connective tubing, a gas source, a monitor, and/or a controller. The controller may be a digital controller, a computer, an electromechanical programmable controller, or any other control system.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. An endotracheal tube comprising:
a conduit having an inner surface defining a fluid passageway;
an adhesion-resistant material that is resistant to microbial adhesion to the conduit disposed on at least a portion of the inner surface, wherein the adhesion-resistant material has a water contact angle of less than 40 degrees; and
an inflatable cuff disposed on the conduit.

2. The endotracheal tube, as set forth in claim 1, wherein the adhesion-resistant material is nonswellable in water.

3. The endotracheal tube, as set forth in claim 1, comprising an adhesion-resistant material disposed on at least a portion of an outer surface of the conduit.

4. The endotracheal tube, as set forth in claim 1, wherein the adhesion-resistant material is compounded with the conduit such that the adhesion-resistant material and the conduit form a single monolithic layer.

5. The endotracheal tube, as set forth in claim 1, wherein the adhesion-resistant material and the conduit comprise a coextrusion.

6. The endotracheal tube, as set forth in claim 5, wherein the adhesion-resistant material comprises at least one of a polyethylene glycol (PEG), a polyvinyl alcohol (PVA), silicone or any combination thereof.

7. The endotracheal tube, as set forth in claim 5, wherein the adhesion-resistant material has a water contact angle of less than 30 degrees.

8. The endotracheal tube, as set forth in claim 1, wherein the adhesion-resistant material has a water contact angle of less than 10 degrees.

9. The endotracheal tube, as set forth in claim 1, wherein the adhesion-resistant material comprises a layer less than 1 mm thick.

10. The endotracheal tube, as set forth in claim 1, comprising a lumen operatively connected to the inner surface of the conduit, wherein the lumen is adapted to dispose the adhesion-resistant material on the inner surface of the conduit.

11. The endotracheal tube, as set forth in claim 1, comprising a ventilator operatively connected to the conduit.

12. A method of manufacturing an endotracheal tube comprising:
providing a conduit having an inner surface defining a fluid passageway;
providing an adhesion-resistant material that is resistant to microbial adhesion disposed on at least a portion of the inner surface, wherein the adhesion-resistant material has a water contact angle of less than 40 degrees; and
providing an inflatable cuff disposed on the conduit.

13. The method, as set forth in claim 12, comprising providing an adhesion-resistant material disposed on at least a portion of an outer surface of the conduit.

14. The method, as set forth in claim 12, wherein providing the adhesion-resistant material comprises compounding the adhesion-resistant material with the conduit such that the adhesion-resistant material and the conduit form a single monolithic layer.

15. The method, as set forth in claim 12, wherein providing the adhesion-resistant material comprises coextruding the conduit and the adhesion-resistant material.

16. The method, as set forth in claim 12, wherein providing the adhesion-resistant material comprises providing a polyethylene glycol (PEG), a polyvinyl alcohol (PVA), silicone or any combination thereof.

17. The method, as set forth in claim 12, wherein providing the adhesion-resistant material comprises providing an adhesion-resistant material with a water contact angle of less than 30 degrees.

18. The method, as set forth in claim 12, wherein providing the adhesion-resistant material comprises providing an adhesion-resistant material with a water contact angle of less than 10 degrees.

19. The method, as set forth in claim 12, wherein the providing the adhesion-resistant material comprises providing the adhesion-resistant material in a layer less than 1 mm thick.

20. The method, as set forth in claim 12, comprising providing a lumen operatively connected to the inner surface of the conduit, wherein the lumen is adapted to dispose the adhesion-resistant material on the inner surface of the conduit.

21. A method of decreasing microbe adhesion to an endotracheal tube comprising:
inserting an endotracheal tube into a patient, wherein the endotracheal tube comprises a conduit having an inner surface defining a fluid passageway; and
applying an adhesion-resistant material on at least a portion of the inner surface, wherein the adhesion-resistant material is adapted to prevent microbe adhesion and wherein the adhesion-resistant material has a water contact angle of less than 40 degrees.

22. The method, as set forth in claim 21, wherein the adhesion-resistant material comprises a carboxycellulose.

23. The method, as set forth in claim 21, applying the adhesion-resistant material comprises comprises applying the adhesion-resistant material in a layer less than 1 mm thick.

24. The method, as set forth in claim 21, wherein applying the adhesion-resistant material comprises injecting the adhesion-resistant material through a lumen operatively connected to the inner surface of the conduit.

25. An endotracheal tube kit comprising:
an endotracheal tube comprising:
a conduit having an inner surface defining a fluid passageway;
an inflatable cuff disposed on the conduit; and
a lumen operatively connected to the inner surface of the conduit, wherein the lumen is adapted to dispose an adhesion-resistant material that is resistant to microbial adhesion on the inner surface of the conduit, wherein the adhesion-resistant material has a water contact angle of less than 40 degrees; and
a syringe comprising the adhesion-resistant material that is resistant to microbial adhesion, wherein the syringe is adapted to be operatively connected to the lumen.
